(19) United States
(12) Patent Application Publication
(10) Pub. No.: US 2021/0370016 A1
(43) Pub. Date: Dec. 2, 2021

(54) RESPIRATORY DEVICE WITH IMPROVED HUMIDIFICATION OF THE RESPIRATION GAS

(71) Applicant: Hamilton Medical AG, Bonaduz (CH)

(72) Inventor: Dino DE-STEFANI, Chur (CH)

(21) Appl. No.: 17/264,079

(22) PCT Filed: Aug. 26, 2019
(86) PCT No.: PCT/EP2019/072731

§ 371 Date: Jan. 28, 2021

(30) Foreign Application Priority Data
Aug. 28, 2018 (DE) 10 2018 214 556.3

(51) Int. Cl. A61M 16/16; A61M 16/10; A61M 16/00

(52) U.S. Cl. CPC A61M 16/161; A61M 16/109; A61M 16/1095; A61M 2016/003; A61M 16/024; A61M 2205/3368; A61M 16/0003

(57) ABSTRACT

The invention relates to a respiratory device (10) for the artificial respiration of a patient (12), comprising: —a respiration gas source assembly (15, 62), —a flow-changing device (16), —a humidifier device (38) which is designed to increase the value of the absolute humidity of the inspiratory respiration gas flow (AF), said humidifier device (38) having a liquid store (40) and an evaporation device (76) with a variable output for this purpose, —a respiration gas line assembly (30), —a proximal temperature sensor (48) which detects the temperature of the respiration gas flow (AF) in the proximal longitudinal end region (30a) of the respiration gas line assembly (30), —a humidity sensor assembly (66) which directly or indirectly detects the absolute humidity of the inspiratory respiration gas flow (AF), —a flow sensor (44), and —a controller (18) which is designed to control the operational output of the evaporation device (76)

This application claims priority to German Application Serial No. DE102018214556.3 filed on Aug. 28, 2018, which is expressly incorporated herein by reference.

The present invention relates to a ventilation apparatus for artificial ventilation of a patient, encompassing:
a respiratory gas source arrangement that furnishes an inspiratory respiratory gas for artificial ventilation of the patient;
a flow modification apparatus that is embodied to generate and to quantitatively modify an inspiratory respiratory gas flow;
a humidification apparatus that is embodied to quantitatively increase the absolute moisture content of the inspiratory respiratory gas flow, the humidification apparatus comprising for that purpose a liquid reservoir and a modifiable-power-level evaporation apparatus;
a respiratory gas conduit arrangement having a proximal longitudinal end located closer to the patient during operation and having a distal longitudinal end located farther from the patient during operation, in order to convey the inspiratory respiratory gas flow from the humidification apparatus to the patient;
a proximal temperature sensor that is embodied to detect the temperature of the respiratory gas flow in the proximal longitudinal end region of the respiratory gas conduit arrangement;
a humidity sensor arrangement that is embodied to detect at least one humidity state value of the inspiratory respiratory gas flow which indirectly or directly represents an absolute moisture content of the inspiratory respiratory gas flow;
a flow sensor that is embodied to quantitatively detect the respiratory gas flow;
a control apparatus which is signal-transferringly connected to the proximal temperature sensor, to the humidity sensor arrangement, and to the flow sensor, and which is embodied to control the operating power level of the evaporation apparatus depending on a predefined setpoint moisture content of the respiratory gas and depending on signals of the proximal temperature sensor, of the humidity sensor arrangement, and of the flow sensor.

A similar ventilation apparatus is known from US 2013/0073013 A1. The known ventilation apparatus uses ambient air as a respiratory gas source, and uses a combination of a fan and valve to quantitatively modify the flow of a respiratory gas flow in the ventilation apparatus. An evaporator/humidification apparatus is provided in order to humidify the respiratory gas as necessary. The respiratory gas flow flows over a water reservoir present in the evaporator/humidification apparatus and thereby picks up water vapor present above the water level of the water reservoir.

The ventilation apparatus known from US 2013/0073013 A1 further encompasses a heatable respiratory gas conduit arrangement to prevent undesired condensation of moisture out of the respiratory gas by delivering heat to the respiratory gas conduit arrangement.

A flow sensor for ascertaining the respiratory gas flow, a temperature sensor for detecting the temperature of the respiratory gas, and a moisture sensor for detecting the moisture content of the respiratory gas are provided downstream from the humidification apparatus of the known ventilation apparatus but before the heated section of the respiratory gas conduit apparatus begins, i.e. in the region of the distal end of the respiratory gas conduit arrangement. Sensors for detecting the ambient temperature and ambient relative humidity are furthermore provided.

Operating parameters of the ventilation apparatus can be adjusted, by a physician or by care personnel, via an input/output apparatus.

A control apparatus of the known ventilation apparatus controls the ventilation apparatus in a manner that is not further characterized. The control apparatus ascertains, from the detected ambient temperature and the detected ambient relative humidity, from the detected temperature of the respiratory gas flow and the detected moisture content of the respiratory gas flow, and further depending on operating data and design data of the respiratory gas conduit arrangement, a setpoint temperature for operation of the heating system of the respiratory gas conduit arrangement. The setpoint temperature is selected in such a way that the temperature in the respiratory gas conduit arrangement is above the respective dew point temperature. The control apparatus controls the heating system of the respiratory gas conduit arrangement depending on the selected setpoint temperature. According to the disclosure of US 2013/0073013 A1, the dew point temperature is obtained from the available psychrometric data, i.e. from the detected temperature and the detected relative moisture content of the respiratory gas flow.

Another ventilation apparatus is known from EP 2 143 459 B1. This too discloses a respiratory gas conduit arrangement having a respiratory gas source and having a fan as a flow modification apparatus. The ventilation apparatus known from EP 2 143 459 B1 likewise comprises an evaporator/humidification apparatus. This too has a water reservoir over which the respiratory gas flow flows during operation, the respiratory gas flow being humidified as it flows over.

That portion of the respiratory gas conduit arrangement of the known ventilation apparatus which extends from the humidification apparatus to the proximal longitudinal end of the respiratory gas conduit arrangement is heatable. In this portion, a flow sensor and a temperature sensor for ascertaining the temperature and the quantitative flow of respiratory gas are arranged in that longitudinal end region of the respiratory gas conduit arrangement which is located closer to the humidification apparatus. A proximal temperature sensor is also arranged at the proximal longitudinal end of the respiratory gas conduit arrangement, at which a patient interface can be attached in order to connect the respiratory gas conduit arrangement to a patient who is to be ventilated.

On a control apparatus of the known ventilation apparatus, an operator can set a desired respiratory gas temperature or a desired moisture level of the respiratory gas supplied by the ventilation apparatus. The control apparatus is data-transferringly connected to the two temperature sensors and to the flow sensor, and thereby receives their detected values.

Using tables, formulas, or characteristics diagrams stored in a data memory, and in accordance with a required respiratory gas moisture content and a respiratory gas flow and the respiratory gas temperature ascertained via sensors, the control apparatus controls the energy delivery to a heating plate of the humidification apparatus so as thereby to furnish a respiratory gas flow having a desired respiratory gas moisture content and a desired respiratory gas temperature. The required respiratory gas moisture content is either the moisture content set by the operator, or a moisture content previously stored in a data memory.

As is commonly known, delivery to the patient of a respiratory gas flow having a correct degree of humidity is particularly important in artificial ventilation. If the delivered respiratory gas is too dry, the patient becomes dehydrated in the course of artificial ventilation. If the delivered respiratory gas is too moist, water can condense out of the respiratory gas and travel in liquid form into the patient's lungs, where it can interfere with desired metabolic gas exchange.

Even if the water condensed out of the respiratory gas does not travel as far as the patient's lungs, it can condense onto walls of the respiratory gas conduit arrangement and/or of sensors, for example a proximal flow sensor, and can thereby reduce the measurement accuracy of the sensors.

The relative humidity of the respiratory gas is more critical here than its absolute humidity. The absolute humidity indicates the quantity of water dissolved in a reference volume of air. The relative humidity is an indication of how much moisture a predetermined reference volume of air can still accept and, for example, extract from the patient, or how close the respiratory gas is to its moisture saturation point. When a respiratory gas is almost saturated with moisture, even small changes in the respiratory gas temperature can result in highly undesirable condensation of liquid out of the respiratory gas (also referred to among specialists as “rain out”).

The object of the present invention is therefore to refine a ventilation apparatus of the kind recited initially in such a way that it can ventilate a patient in as precise and stable a manner as possible, over a long ventilation period, with correctly humidified respiratory gas.

This object is achieved according to the present invention by a ventilation apparatus as described at the beginning of the Application, in which additionally the humidity sensor arrangement is arranged upstream, in an inspiration direction of the respiratory gas flow, from the humidification apparatus and is embodied to detect the at least one humidity state value upstream from the humidification apparatus; the ventilation apparatus comprising at least one ambient temperature sensor that is embodied to detect the temperature of the environment of the ventilation apparatus; the control apparatus being signal-transferringly connected to the at least one ambient temperature sensor and being embodied to control the operating power level of the evaporation apparatus, in addition to the dependences already recited, additionally depending on signals of the at least one ambient temperature sensor.

As a result of the arrangement of the humidity sensor arrangement upstream, in an inspiration direction, from the humidification apparatus, the humidity sensor arrangement can detect the humidity of the respiratory gas in the initial state, i.e. before any humidification action. The control apparatus can thereby ascertain the humidify requirement of the respiratory gas particularly accurately.

This is, surprisingly, more advantageous than the arrangement, known from US 2013/0073013 A1, of the humidity sensor arrangement downstream from the humidification apparatus, which one would expect to enable feedback regulation of the respiratory gas moisture content based on the detected value of the humidity sensor arrangement. In actuality, however, a change in the respiratory gas moisture content resulting from a change in the energy delivery to a heating plate of the humidification apparatus occurs very slowly and sluggishly, so that a control loop with increasingly small offsets between the setpoint humidity and actual humidity becomes more and more difficult to implement with sufficient accuracy.

It has therefore become apparent that for regulation of the respiratory gas moisture content it is more advantageous to detect the respiratory gas moisture content before the respiratory gas reaches the humidification apparatus, so as thereby to ascertain the actual humidification requirement and to operate the humidification apparatus in accordance with that requirement. This causes the actual respiratory gas moisture content to approach the desired setpoint moisture content more strongly and more quickly than any regulation based on detection of the respiratory gas moisture content after the humidification apparatus.

As a result of detection of the ambient temperature with the ambient temperature sensor, the control apparatus has available to it a variable that is an indicator of thermal losses of the humidification apparatus and/or of the respiratory gas to the environment. It is thereby possible for the control apparatus not only to ascertain the operating power level of the evaporation apparatus based on the immediate operating parameters, such as the respiratory gas flow, the temperature of the respiratory gas at the proximal temperature sensor, and the respiratory gas moisture content, but also to take into account, for determination of that operating power level, any losses to the environment.

The control apparatus is therefore embodied to use the ambient temperature detected by the ambient temperature sensor as a correction variable for correction of the ascertained operating power level of the evaporation apparatus in terms of any thermal ambient losses to the external environment.

Correction of the operating power level delivered to the evaporation apparatus by the control apparatus in terms of thermal ambient losses is particularly important specifically for emergency medicine and first aid. Ventilation apparatuses such as the one discussed here are used not only in clinical settings in which parameters are predictable, but also in emergency transport vehicles, including transport helicopters, whose environmental conditions are anything but constant and predictable. To cite an extreme case: consider an emergency physician engaged in mountain rescue in a transport helicopter, whose task, in winter in an Alpine environment, is to rescue severely injured accident victims at elevations between 10,000 and 16,000 feet above sea level, and to be able to immediately provide life-sustaining care.

The control apparatus can control the operating power level of the evaporation apparatus on the basis of the aforesaid parameters based on empirically ascertained correlations that have been ascertained previously in the laboratory for a plurality of parameter combinations. The correlations can be stored, as a formula correlation, a characteristics diagram, and/or a table, in a data memory that can be queried by the control apparatus.

The respiratory gas moisture content can be adjusted by the control apparatus in several steps. Firstly, the control apparatus can ascertain a setpoint respiratory gas moisture content based on a desired respiratory gas temperature at the measurement location of the proximal temperature sensor (hereinafter also referred to as the “proximal respiratory gas temperature”) and further based on a desired quantitative respiratory gas flow. This determination can in turn can be made on the basis of empirically ascertained data correlations that are stored in a data memory that can be queried by the control apparatus. Based on the setpoint respiratory gas moisture content that is thereby determined and constitutes the setpoint variable, the operating power level of the evaporation apparatus can then be ascertained, again based on empirically determined correlations that are stored in a data memory, on the basis of the actual respiratory gas moisture content detected upstream from the ventilation apparatus and on further ones of the aforesaid parameters.

The ambient temperature, constituting a loss correction variable, can be taken into account directly upon ascertainment of the operating power level, or an operating power level can be ascertained based on the parameters recited earlier and can then be corrected in accordance with the detected ambient temperature.

As stated above, in terms of the physiological tolerability of ventilation, the relative humidity of the respiratory gas is more critical than the absolute humidity. The absolute humidity of the respiratory gas is more suitable for ascertaining the operating power level of the evaporation apparatus, however, since the mass of water contained in a predetermined volume of respiratory gas is known per se. It is very easy to ascertain therefrom the differential mass of water that must be delivered to the respiratory gas in order for the respiratory gas to be correctly humidified. The ascertainable power level necessary for evaporation of a known mass of water within a predetermined time is then the operating power level that is as yet uncorrected in terms of ambient losses. The relative humidity of the respiratory gas considered of itself, conversely, would denote only the ratio of the current respiratory gas moisture content to the saturated respiratory gas moisture content, but the unknown differential quantity of water would not be ascertainable therefrom.

The absolute moisture content of the respiratory gas is equal to its relative moisture content, however, given a simultaneous knowledge of the respiratory gas temperature on the one hand and the dew point curve of the liquid to be evaporated (as a rule, water) on the other hand. The humidity sensor arrangement can therefore either comprise a sensor for ascertaining the absolute respiratory gas moisture content or a sensor for ascertaining the relative respiratory gas moisture content, and can comprise a temperature sensor provided the dew point curve of the liquid to be evaporated, or a data correlation corresponding to the dew point curve, is stored in a data memory that can be queried by the control apparatus.

Sensor-based ascertainment of an indicated parameter is equivalent, for purposes of the present application, to direct ascertainment of a variable other than the indicated parameter if that other variable represents the indicated parameter on the basis of a known correlation, for example a calibration correlation.

The terms “moisture (content)” and “humidity” are used synonymously in the present Application.

The empirical ascertainment of data correlations does represent an outlay, since the aforesaid influencing parameters must be varied in controlled fashion in a laboratory in the context of their expected operating value range, and detected in terms of their influence on the target parameter. For ventilation apparatuses of the same design, however, that outlay needs to be made only once. The data correlations thereby ascertained for one design configuration of ventilation apparatuses can then be used for a plurality of ventilation apparatuses of the same design. It is then sufficient to check on a random basis as to whether or not a previously ascertained data correlation is correct for a specific ventilation apparatus.

The flow sensor can then detect the magnitude at least of the inspiratory respiratory gas flow, and the proximal temperature sensor can detect the temperature, in the proximal longitudinal end region of the respiratory gas conduit arrangement, so that humidification of the respiratory gas is effected on the basis of parameters that are as close as possible to the patient.

In order to define a desired ventilation situation, the ventilation apparatus can comprise an input apparatus for inputting at least one setpoint temperature of the respiratory gas flow in the proximal longitudinal end region of the respiratory gas conduit arrangement and/or for inputting at least one setpoint moisture content of the respiratory gas and/or for inputting at least one setpoint respiratory gas flow. It is also possible for only some of the aforementioned parameters to be inputtable at the input apparatus, while others of the aforementioned parameters can be ascertained on the basis of empirically ascertained data correlations in accordance with the inputted parameters and/or further ones.

In order to simplify the operation of the ventilation apparatus, for example, it can comprise an input apparatus for inputting a setpoint temperature of the respiratory gas flow in the proximal longitudinal end region of the respiratory gas conduit arrangement, and can furthermore comprise a data memory which can be queried by the control apparatus and in which a (preferably empirically ascertained) correlation between temperature values of the respiratory gas flow in the proximal longitudinal end region of the respiratory gas conduit arrangement and associated setpoint moisture values of the respiratory gas is stored. All that is then necessary in order for the control apparatus to be capable of determining the associated setpoint moisture content of the respiratory gas is to input the proximal respiratory gas flow temperature.

In order to avoid undesired condensation of liquid onto the walls of the respiratory gas conduit arrangement, at least one inspiratory conduit portion of the respiratory gas conduit arrangement can comprise a conduit heating apparatus. The inspiratory conduit portion can be heated with this conduit heating apparatus. It is thereby possible to prevent the temperature of a wall of the inspiratory conduit portion from falling below the dew point temperature of the respiratory gas flowing in the conduit portion. The control apparatus can then be embodied to control the operating power level of the conduit heating apparatus depending on a predefined setpoint temperature of the respiratory gas flow in the proximal longitudinal end region of the respiratory gas conduit arrangement. The control apparatus can furthermore take the detected ambient temperature into account when ascertaining the energy or power level to be delivered to the conduit heating apparatus. This considerably simplifies the control loop for heating the conduit portion, since as a rule the respiratory gas conduit arrangement is exposed on its outer side to ambient air at ambient temperature. Undesired temperature fluctuations on the heated wall of the inspiratory conduit portion can be avoided if the ambient temperature is taken into account when heating the conduit portion. The temperature of the heating conduit portion can thereby be maintained more consistently.

The respiratory gas source arrangement, constituting a respiratory gas source, can comprise an intake opening through which ambient air, or gas from a predetermined gas reservoir, can be taken in. Additionally or alternatively, the respiratory gas source arrangement can comprise a gas reservoir as a respiratory gas source, for example a reservoir container or a connecting configuration for attachment of a supply conduit that connects the ventilation apparatus to a locally installed gas reservoir, as is often the case in hospitals. In order to furnish the capability of mixing different gases to yield a respiratory gas, the respiratory gas source arrangement can comprise a plurality of individual respiratory gas sources such as those recited above. The different gases to be mixed can be at different temperatures and/or can have different moisture contents as a result of expansion and how they are furnished. In order to ensure that the humidity sensor arrangement detects the moisture content of the respiratory gas actually delivered to the patient, the humidity sensor arrangement is preferably arranged downstream, in an inspiration direction of the respiratory gas flow, from the respiratory gas source arrangement. For the reasons recited, it is particularly preferred that no further respiratory gas constituent be added, downstream from the humidification apparatus in an inspiration direction, to the respiratory gas emerging from the humidification apparatus.

The control apparatus can be signal-transferringly connected to the evaporation apparatus in order to detect and control the energy or power level delivered to the evaporation apparatus. Additionally or alternatively, the control apparatus can be signal-transferringly connected to an energy supply of the evaporation apparatus. For example, the control apparatus can be embodied to directly detect an electrical current delivered to the evaporation apparatus, and the electrical voltage applied in that context. If embodiment of the control apparatus for the direct detection of energy, in particular electrical energy, implies too great an outlay, the control apparatus can be signal-transferringly connected to at least one energy sensor that is embodied to detect an energy delivered to the evaporation apparatus. The energy and/or power level currently being delivered to the evaporation apparatus can be ascertainable by the control apparatus from signals that are transferred to the control apparatus via the signal-transferring connection. The ascertainment of energy and that of power level differ only in that the power level represents a detection of energy over time.

In order to allow the transfer of respiratory gas, from the ventilation apparatus to the particular patient being ventilated, to be adapted in demand-compatible fashion to the patient's needs, the respiratory gas conduit arrangement can comprise at its proximal end a coupling configuration for coupling the respiratory gas conduit arrangement to a patient interface that transfers respiratory gas between the respiratory gas conduit arrangement and the patient. A patient interface of this kind can be an endotracheal tube, a larynx mask, or a combination tube, and the like. The coupling configuration can be embodied on a proximal flow sensor that is provided at the longitudinal end of the respiratory gas conduit arrangement in order to detect the respiratory gas flow quantitatively.

Even when the respiratory gas conduit arrangement is heatable in portions or entirely, the patient interface couplable to the respiratory gas conduit arrangement as a rule is not heatable. The temperature of the patient interface and of the respiratory gas flow in the patient interface can therefore fluctuate more than the temperature of the respiratory gas conduit arrangement and of the respiratory gas flow therein.

Regardless of whether the patient interface is a tube or a mask, a portion of the patient interface which directly adjoins the coupling configuration of the respiratory gas conduit arrangement will always be surrounded by ambient air, and can thereby be influenced by the ambient temperature.

Specifically in the emergency transport situation described above, the ambient temperature can be considerably lower than the patient's body temperature and than a desired temperature of the respiratory gas flow. Condensation of moisture out of the respiratory gas may therefore occur at the patient interface, for example because the temperature at that wall of the patient interface which is cooled by the ambient air has fallen below the dew point temperature of the respiratory gas flow. Because the patient interface is located particularly close to the patient, and in the case of a tracheal tube is in fact introduced into the patient's body, condensation of liquid in the region of the patient interface is a particular hazard for the patient being ventilated.

Condensation can take place on walls of the patient interface which are greatly cooled by the ambient air. Additionally or alternatively, condensation can take place as fogging directly in the respiratory gas flow.

Not only is the patient interface as a rule unheated; it is as a rule a single-use or disposable item, and is also not fitted with sensors. This makes it difficult to recognize and counteract condensation in the patient interface.

In order to avoid undesired condensation of moisture in the respiratory gas flow in the patient interface, the control apparatus can be embodied to ascertain, depending on operating parameters of the respiratory gas flow such as temperature, mass flow or volumetric flow, and/or relative humidity, a resultant state of the ventilation situation downstream, in an inspiration direction, from the coupling configuration, and to modify the operating power level of the evaporation apparatus depending on the ascertained result. If a resultant state that is critical in terms of condensation in the patient interface is recognized by the control apparatus, the operating power level of the evaporation apparatus is modified, as a rule reduced, and the risk of condensation in the patient interface is thereby decreased or in fact eliminated.

For example, the operating parameters of the ventilation apparatus which are relevant for ascertaining the resultant state can encompass the temperature of the respiratory gas flow in the proximal longitudinal end region of the respiratory gas conduit arrangement, as detected by the proximal temperature sensor and transferred to the control apparatus, and the ambient temperature as detected by the ambient temperature sensor and transferred to the control apparatus. Additionally or alternatively, the magnitude of the respiratory gas flow ascertained by the flow sensor can be utilized in order to ascertain the resultant state. A further relevant operating parameter can be the nature of the patient interface and its design configuration.

At least one, preferably empirically ascertained, resultant data correlation can in turn be stored in a data memory that can be queried by the control apparatus, said correlation associating a set of operating parameters of the ventilation apparatus (which can be several or all of the operating parameters recited above) with a resultant parameter that indicates an occurrence of condensation of respiratory gas moisture in the patient interface.

The resultant parameter can be a binary parameter that either can assume a first value that indicates that no condensation is occurring in the patient interface, or can assume a second value that indicates the opposite, namely that condensation is taking place in the patient interface.

Depending on the resultant data correlation stored in the data memory, for example utilizing a modeled mapping (which can be computationally handled analytically or numerically) of the ventilation situation at the interface, the resultant parameter can indicate a probability with which condensation of liquid from the respiratory gas flow is predicted to take place in the patient interface.

A probability value of this kind can additionally or alternatively be produced by the fact that in the context of empirical ascertainment of the data correlation, among the operating parameters relevant to the existing ventilation situation, condensation has taken place only in a portion of the empirically investigated cases involving identical parameters, or by the fact that a data correlation does not exist for all the operating parameter values that describe the existing ventilation situation, for instance because the data correlation is defined in tabular form based on interpolation points. In this case, missing values can be interpolated and/or extrapolated from the values that are present. In the context of a complex multidimensional correlation between operating parameters and a resultant parameter, uncertainties that can occur in the determination of the resultant parameter can appropriately be reproduced or interpreted as a probability.

The control apparatus can thereby, in accordance with the resultant parameter, modify, in particular decrease, the operating power level of the evaporation apparatus in order to avoid impending condensation in the patient interface. In the case of the aforementioned simple binary resultant parameter, for example, the one value can cause a decrease in the operating power level of the evaporation apparatus, but the other value does not. In the case of the above-described resultant parameter in the form of a probability value, i.e. a value between 0 and 1 or between 0% and 100%, a threshold probability value, for instance a probability of 40% or more, of condensation in the patient interface can be predetermined, exceedance of which by the resultant parameter leads to a decrease in the operating power level of the evaporation apparatus by the control apparatus. With or without a threshold probability value, the magnitude of the decrease in the operating power level can depend on the magnitude of the probability with which the resultant parameter forecasts condensation in the patient interface. The dependence of the magnitude of the decrease in the operating power level on the probability value of the resultant parameter can be linear or can be progressive, so that starting from an existing operating power level, the decrease in the operating power level in accordance with the resultant parameter increases with increasing condensation probability more greatly than does the condensation probability.

In a preferred application instance that is easy to handle computationally but is nevertheless highly effective, the resultant state or resultant parameter of the ventilation situation can encompass a resultant temperature of the conduit wall of the patient interface and/or of the respiratory gas flow in the patient interface.

A first advantage of using a resultant temperature to describe the resultant state is on the one hand that, assuming a known design configuration of the patient interface, including the materials used to manufacture the patient interface, and assuming known environmental and operating conditions based on the most recent sensor-detected operating state, for instance in the region of the coupling configuration at the proximal longitudinal end of the respiratory gas conduit arrangement, resultant temperatures downstream (in an inspiration direction) from the coupling configuration can be calculated with good accuracy based on thermodynamic models. It is thus possible either to calculate a resultant temperature in the patient interface, based on thermodynamic models and proceeding from parameters detected instrumentally, in particular parameters located immediately next to the patient interface; or to read out a resultant temperature based on empirically developed characteristics diagrams based on the aforesaid parameters; or a combination of model prediction and readout from empirical data correlations can be used to ascertain the resultant temperature.

A second advantage of using a resultant temperature to describe the resultant state is that the resultant temperature alone can be sufficient for predicting condensation of liquid out of the respiratory gas flow. If the absolute moisture content of the respiratory gas flow is known, whether by direct measurement or by measuring the relative moisture content and the temperature of the respiratory gas flow, the dew point temperature of the respiratory gas flow is then known or is ascertainable from stored dew-point curves. If the resultant temperature is below the dew point temperature ascertained for the respective respiratory gas flow, condensation—i.e. undesired “rain out”—of moisture in the respiratory gas in the patient interface is preponderantly probable.

As already indicated above, for prediction of a resultant temperature in the patient interface the ventilation apparatus can comprise a data memory which can be queried by the control apparatus and in which operating parameter values detectable instrumentally by the ventilation apparatus, for instance the ambient temperature, and/or operating parameter values of the respiratory gas flow which are detectable in the respiratory gas conduit arrangement, are associated with resultant temperature values of the conduit wall of the patient interface and/or of the respiratory gas flow. Merely for the sake of completeness and clarity, be it noted that for purposes of the present Application, a value interpolated or extrapolated from instrumentally detected operating parameter values is also considered to be instrumentally detected.

As a result of the data correlation stored in the aforesaid data memory, whether as an empirically ascertained data table or data characteristics diagram, or as an analytical or numerical calculation model, the control apparatus can ascertain the resultant state with good accuracy depending on: the temperature, detected by the proximal temperature sensor, of the respiratory gas flow in the proximal longitudinal end region of the respiratory gas conduit arrangement; the magnitude of the respiratory gas flow detected by the flow sensor; and the predefined setpoint moisture content of the respiratory gas. The probability of undesired condensation in the patient interface can thus be predicted very quickly, with good accuracy, by way of a manageable number of known or instrumentally detectable parameter values.

Undesired condensation of moisture in the respiratory gas in the patient interface can be predicted particularly accurately when the control apparatus ascertains the resultant state depending on the ambient temperature detected by the ambient temperature sensor. The ambient temperature, which acts from outside on at least a portion of the patient interface, is a factor that greatly influences the temperature of the respiratory gas flowing in the patient interface, and therefore influences possible condensation in the patient interface.

Because the effects on the patient can be that much more disadvantageous the farther downstream, i.e. the closer to the patient, the condensation in the respiratory gas in the patient interface takes place so that liquid in the patient interface becomes a risk, according to an advantageous refinement of the present invention provision is made that the control apparatus ascertains the resultant state for an ascertainment location located closer to the proximal end of the patient interface than to its distal end, the control apparatus preferably ascertaining the resultant state for the proximal end of the patient interface.

Additionally or alternatively, the control apparatus can ascertain the resultant state for a portion of the patient interface which is located between the coupling configuration and the entry point of the patient interface into the body of the patient. The resultant state, in particular the resultant temperature, is then ascertained for precisely that portion of the patient interface which is exposed directly to the influence of the environment and thus of the ambient temperature, which in extreme operating situations can deviate greatly from the desired temperature of the respiratory gas flow.

When an inspiratory conduit portion of the respiratory gas conduit arrangement is heatable by way of a conduit heating arrangement, the control apparatus additionally can modify, in particular can raise, the operating power level of the conduit heating arrangement depending on the ascertainment result. When assessing whether respiratory gas that is briefly slightly too warm, or contains liquid phase components, is being delivered to the patient, the option of the elevated respiratory gas temperature is to be preferred.

The coupling configuration is preferably embodied at the proximal longitudinal end of a Y-conduit portion of the respiratory gas conduit arrangement, an inspiration hose and an expiration hose being respectively attached to the two distal longitudinal ends of the Y-conduit portion.

The flow sensor can be a distal flow sensor that is received in the operating housing of the ventilation apparatus. Additionally or alternatively, the flow sensor can be a proximal flow sensor that is arranged, preferably replaceably, at the proximal longitudinal end of the respiratory gas conduit arrangement. The proximal flow sensor can be arranged between the coupling configuration and the patient interface. A proximal flow sensor of this kind can offer high measurement accuracy, and can nevertheless be inexpensive, if it is embodied as a differential pressure flowthrough sensor. The proximal flow sensor can be part of the coupling configuration and can itself be embodied for coupling of the patient interface.

The evaporation apparatus preferably comprises a heating apparatus with which the liquid reservoir is heated and evaporated. The control apparatus is then preferably embodied to decrease the heating output of the heating apparatus when the aforementioned ascertainment result indicates that condensation of liquid in the patient interface is certain or is preponderantly probable, or is above a predefined probability threshold, or that a resultant temperature is above a predefined resultant temperature threshold.

Alternatively or additionally, the evaporation apparatus can be an ultrasonic nebulizer. In such circumstances, the operating power level that is modifiable by the control apparatus depending on the ascertainment result is the operating power level of the ultrasonic nebulizer. The conditions for decreasing the nebulization power level are the conditions recited above for decreasing the heating power level.

The present invention will be explained in further detail below with reference to the appended drawings, in which:

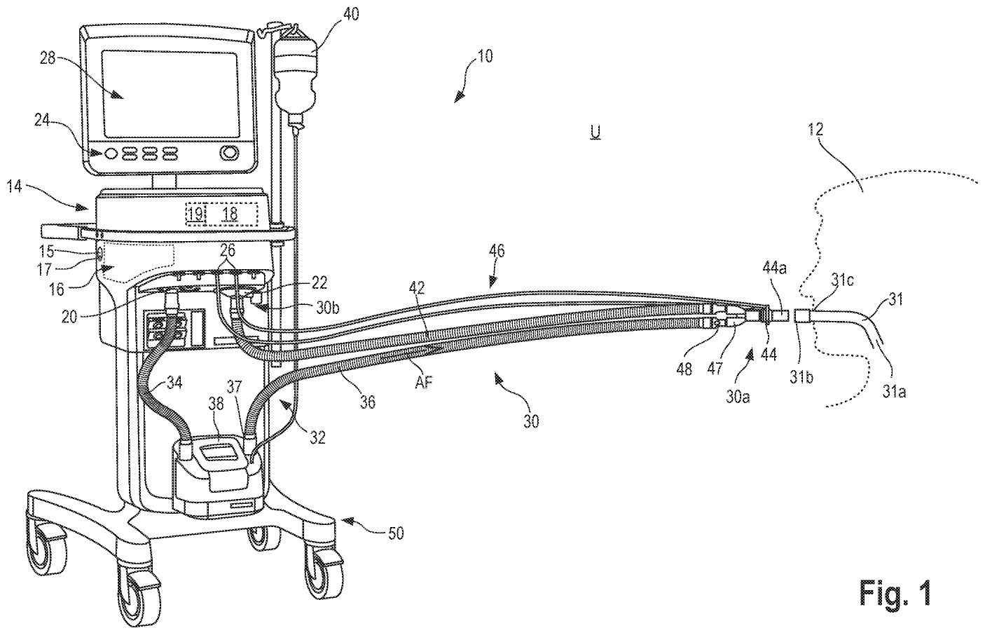

FIG. 1 schematically depicts a ventilation apparatus according to the present invention, configured for artificial ventilation of a patient; and

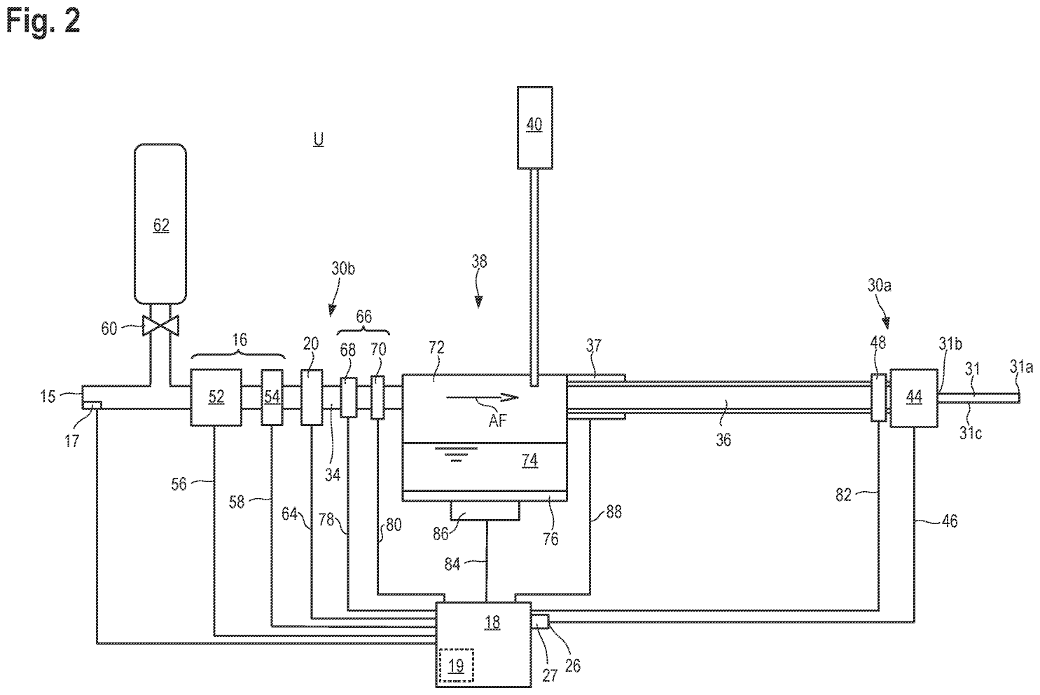

FIG. 2 schematically depicts the inspiratory respiratory gas conduit branch of the ventilation apparatus of FIG. 1.

In FIG. 1, an embodiment according to the present invention of a ventilation apparatus is labeled in general with the number 10. Ventilation apparatus 10 serves, in the example depicted, for artificial ventilation of a human patient 12.

Ventilation apparatus 10 comprises a housing 14 in which an intake opening 15 is embodied and in which a flow modification apparatus 16 and a control device 18 (not visible from outside because of the opaque housing material) are received. Intake opening 15 allows flow modification apparatus 16 to take in ambient air from external environment U of the ventilation apparatus and, after purification known per se by means of filters, to deliver it as respiratory gas to patient 12. Intake opening 15 is therefore a respiratory gas source arrangement for purposes of the present Application.

An ambient temperature sensor 17, which measures the temperature of the air of environment U and transfers it to sensor apparatus 18, is located in intake opening 15.

Flow modification apparatus 16 is constructed in a manner known per se and can comprise a pump, a compressor, a fan 52, a pressure vessel, a reduction valve 54 (see FIG. 2), and the like. Ventilation apparatus 10 furthermore comprises, in a manner known per se, an inspiration valve 20 and an expiration valve 22.

Control device 18 is usually implemented as a computer or microprocessor. It encompasses a data memory, labeled 19 in FIG. 1, so that data necessary for the operation of ventilation apparatus 10 can be stored and retrieved as necessary. In a network operation context, data memory 19 can also be located outside housing 14 and can be connected to control device 18 by a data transfer connection. The data transfer connection can be constituted by a cable link or a radio link. In order to prevent disruptions of the data transfer connection from having an effect on the operation of ventilation apparatus 10, however, data memory 19 is preferably integrated into control device 18 or at least received in the same housing 14 as that device.

Ventilation apparatus 10 comprises an input apparatus 24, which in the example depicted in FIG. 1 is represented by a keyboard, for inputting data into ventilation apparatus 10 or more precisely into control device 18. As will be explained in further detail below, the keyboard is not the only data input of control device 18. Control device 18 can in fact obtain data through a variety of data inputs, for instance via a network line, a radio link, or via sensor terminals 26 that will be discussed in detail below.

Ventilation apparatus 10 can comprise an output device 28, in the example depicted a display screen, for outputting data to the therapist providing care.

For artificial ventilation, patient 12 is connected to ventilation apparatus 10, more precisely to flow modification apparatus 16 in housing 14, via a respiratory gas conduit arrangement 30. Patient 12 is intubated for that purpose by means of an endotracheal tube constituting a patient interface 31. A proximal longitudinal end 31a of patient interface 31 discharges inspiratory respiratory gas flow AF into the lungs of patient 12. The expiratory respiratory gas flow also flows through proximal longitudinal end 31a into the respiratory gas conduit arrangement.

A distal longitudinal end 31b of patient interface 31 is embodied for connection to respiratory gas conduit arrangement 30. From location 31c downstream (in an inspiration direction) to proximal longitudinal end 31a, the patient interface is surrounded by the body of patient 12. This means conversely that from its distal longitudinal end 31b to location 31c, patient interface 31 is exposed to external environment U and is in (predominantly convective) thermally transferring communication therewith.

Ventilation conduit arrangement 30 comprises an inspiration hose 32 through which fresh respiratory gas can be directed from flow modification apparatus 16 into the lungs of patient 12. Inspiration hose 32 can be interrupted, and can comprise a first inspiration hose 34 and a second inspiration hose 36 between which a humidification apparatus 38, for controlled humidification and optionally also temperature control of the inspiratory respiratory gas delivered to patient 12, can be provided. Humidification apparatus 38 can be connected to an external liquid reservoir 40 by way of which water for humidification, or also a medication e.g. to inhibit inflammation or to dilate the airways, can be delivered to humidification apparatus 38. When the present ventilation apparatus 10 is used as an anesthesia ventilation apparatus, it is thereby possible to deliver volatile anesthetics to patient 12 in controlled fashion via ventilation apparatus 10. Humidification apparatus 38 ensures that the fresh respiratory gas is conveyed to patient 12 with a predetermined moisture content, optionally with addition of a medication aerosol, and at a predetermined temperature.

In the present example, second inspiration hose 36 is electrically heatable by a conduit heating apparatus 37. Conduit heating apparatus 37 can be controlled by control apparatus 18 for operation. Unlike what is said above, first inspiration hose 34 can also be heatable, and/or the at least one hose 34 and/or 36 can be heatable by a conduit heating apparatus 37 that is other than electric, for instance by having a heat exchange medium flow around it.

Respiratory gas conduit arrangement 30 further comprises, in addition to the aforementioned inspiration valve 20 and expiration valve 22, an expiration hose 42 by way of which metabolized respiratory gas is expelled from the lungs of patient 12 into external environment U.

At distal longitudinal end 30b of respiratory gas conduit arrangement 30, inspiration hose 32 is coupled to inspiration valve 20, and expiration hose 42 to expiration valve 22. Preferably only one of the two valves is respectively open simultaneously for passage of a gas flow. Actuation control of valves 20 and 22 is likewise accomplished by control device 18.

During a ventilation cycle, firstly expiration valve 22 is closed and inspiration valve 20 is opened for the duration of the inspiration phase, so that fresh inspiratory respiratory gas can be directed from housing 14 to patient 12. A flow of fresh respiratory gas is produced by flow modification arrangement 16 by controlled elevation of the pressure of the respiratory gas. As a result of the pressure elevation, the fresh respiratory gas flows into the lungs of patient 12 where it expands the body region in the vicinity of the lungs, i.e. in particular the thorax, against the individual elasticity of the body parts near the lungs. The gas pressure in the interior of the lungs of patient 12 also rises as a result.

At the end of the inspiration phase, inspiration valve 20 is closed and expiration valve 22 is opened. The expiration phase begins. Because the gas pressure of the respiratory gas present in the lungs of patient 12 has been elevated until the end of the inspiration phase, said gas flows into external environment U after expiration valve 22 is opened, while the gas pressure in the lungs of patient 12 decreases as the flow continues. When the gas pressure in lungs 12 reaches a positive end expiration pressure (PEEP) that is set on ventilation apparatus 10, i.e. a pressure slightly higher than atmospheric pressure, the expiration phase is terminated with the closing of expiration valve 22, and is followed by a further ventilation cycle.

The so-called ventilation tidal volume, i.e. the volume of respiratory gas for each breath, is delivered to patient 12 during the inspiration phase. The ventilation tidal volume multiplied by the number of ventilation cycles per minute, i.e. multiplied by the ventilation frequency, equals the volume per minute of artificial ventilation being performed in the present case.

Ventilation apparatus 10, in particular control device 18, is preferably embodied to repeatedly update or ascertain, during ventilation operation, ventilation operating parameters that characterize the ventilation operation of ventilation apparatus 10, in order to ensure that ventilation operation is coordinated as optimally as possible, at every point in time, with patient 12 who is respectively to be ventilated. Particularly advantageously, the determination of one or several ventilation operation parameters is made at the ventilation frequency, so that ventilation operating parameters that are current, and are thus optimally adapted to patient 12, can be furnished for each ventilation cycle.

Ventilation apparatus 10 can be data-transferringly connected for that purpose to one or several sensors that monitor the status of the patient and/or the operation of ventilation apparatus 10. A proximal flow sensor 44, which quantitatively detects the respiratory gas flow existing in respiratory gas conduit arrangement 30, is mentioned in FIG. 1 merely as an example of a number of possible sensors. Proximal flow sensor 44, preferably embodied as a differential pressure sensor, can be coupled by means of a sensor lead arrangement 46 to data inputs 26 of control device 18. Sensor lead arrangement 46 can, but does not need to, encompass electrical signal transfer leads. It can also comprise hose lines that transfer the gas pressure, existing on either side of flow sensor 44 in a flow direction, to data inputs 26, where those pressures are quantified by pressure sensors (depicted only in FIG. 2).

More precisely, in the preferred exemplifying embodiment respiratory gas conduit arrangement 30 comprises at its proximal longitudinal end region 30a a separately embodied Y-conduit portion 47, which is connected at its distal end region to second inspiration hose 36 and to expiration hose 42, and which is connected at its proximal end region to proximal flow sensor 44.

Proximal flow sensor 44 comprises at its proximal end region a coupling configuration 44a with which patient interface 31, which could also be a mask rather than a tube, is couplable to proximal flow sensor 44 and consequently to respiratory gas conduit arrangement 30.

Second inspiration hose 36 comprises, at its proximal longitudinal end region, a proximal temperature sensor 48 that measures the temperature of respiratory gas flow AF in second inspiration hose 36 as close as possible to patient 12, and transfers it to control apparatus 18.

Merely for the sake of completeness, be it noted that ventilation apparatus 10 according to the present invention can constitute a mobile ventilation apparatus 10 and can be received on a rollable frame 50.

FIG. 2 is a schematic view of the inspiratory conduit branch of ventilation apparatus 10 of FIG. 1.

Flow modification apparatus 16 encompasses a fan 52 and a reduction valve 54 located downstream therefrom in an inspiration direction. It is also possible for only fan 52 to be provided. For operation, control apparatus 18 can apply control to fan 52 and to reduction valve 54 via respective leads 56 and 58.

Fan 52 can take in ambient air from external environment U through intake opening 15. Additionally or alternatively, gas, for instance pure oxygen, from a reservoir vessel 62 can be used via a valve 60 as a respiratory gas or can be mixed into the ambient air that is taken in. In the exemplifying embodiment depicted, intake opening 15 and reservoir vessel 62 therefore together constitute a respiratory gas source arrangement.

The ambient air that is taken in is purified in ventilation apparatus 10 in a manner known per se, in filters that are not depicted.

Inspiration valve 20, which is controllable by control apparatus 18 via a lead 64 for opening and closing, is located downstream (in an inspiration direction) from flow modification apparatus.

Farther downstream in an inspiration direction, inspiration valve 20 is followed by first inspiration hose 34.

Downstream in an inspiration direction from ventilation apparatus 38 in the narrower sense, ventilation apparatus 10 encompasses a humidity sensor arrangement 66 that, in the economically preferred instance depicted, encompasses a sensor 68 for ascertaining the relative humidity of inspiratory respiratory gas flow AF and a temperature sensor 70 for ascertaining the respiratory gas temperature in the region in which humidity is detected by sensor 68. Alternatively, humidity sensor arrangement 66 could encompass only one sensor for detecting the absolute moisture content of the respiratory gas, but for cost reasons this is not preferred.

Humidity sensor arrangement 66 detects, along with the relative moisture content and the temperature of the respiratory gas upstream from humidification apparatus 48, the relative moisture content and the temperature of the respiratory gas after respiratory gas sources 15 and 62 but before humidification measures are taken by ventilation apparatus 10. Humidity sensor arrangement 66 can be arranged in the housing, shown in FIG. 1, of humidification apparatus 38, but upstream from humidification apparatus 38, which is constituted by a humidification chamber 72 having a liquid reservoir 74 to which thermal energy for evaporating liquid can be delivered via a thermal evaporation apparatus 76 in the form of a heating plate.

The detected values of sensors 68 and 70 are transferrable via leads 78 and 80 to control apparatus 18.

From the detected values thereby obtained for the relative moisture content and the temperature of the respiratory gas before reaching humidification apparatus 38, control apparatus 18 can ascertain the absolute moisture content of the respiratory gas and compare it to a desired setpoint moisture content of the respiratory gas.

The setpoint moisture content of the respiratory gas can be obtained, for example, from a data correlation, stored in data memory 19, of signals of proximal temperature sensor 48, and optionally also of the proximal respiratory gas flow detected by proximal flow sensor 44. Alternatively, a desired setpoint moisture content of the respiratory gas can also be inputted manually via input apparatus 24.

Based on the setpoint moisture content of the respiratory gas, and also depending on the signals of proximal temperature sensor 48 (which are transferred via a lead 82 to control apparatus 18), of humidity sensor arrangement 66, and of proximal flow sensor 44, control apparatus 18 ascertains an operating power level that is to be delivered to evaporation apparatus 76 in the form of a heating plate. Control apparatus 18 applies control via a lead 84 to an energy supply 86 of evaporation apparatus 76 in accordance with the ascertained operating power level. By way of lead 84, control apparatus 18 receives information regarding the operating power level delivered to evaporation apparatus 76, for example in the form of the voltage applied to evaporation apparatus 76 and the current intensity flowing to it. Energy supply apparatus 86 can deliver a variable power level to evaporation apparatus 76, for example by pulse width modulation. In a context of pulse width modulation, the voltage applied to evaporation apparatus 76 is a time-averaged voltage.

Liquid is evaporated from liquid reservoir 74 by evaporation apparatus 76, and is entrained by respiratory gas flow AF that flows over liquid reservoir 74.

In order to account for heat losses from evaporation apparatus 76 to environment U, control apparatus 18 ascertains the energy to be delivered per unit time to evaporation apparatus 76 via energy supply apparatus 86 additionally in consideration of the ambient temperature ascertained by ambient temperature sensor 17.

In order to ascertain the operating power level to be delivered to evaporation apparatus 76 depending on the setpoint moisture content of the respiratory gas, and further depending on the proximal respiratory gas temperature, the absolute moisture content detected indirectly by humidity sensor arrangement 66, the proximal respiratory gas flow detected by proximal flow sensor, and further depending on the ambient temperature, a corresponding data correlation, which was determined previously in the laboratory for a ventilation apparatus 10 of the same design by parameter variation under controlled conditions, is stored in data memory 19.

It is therefore to be assumed that as a result of the operation of evaporation apparatus 76, controlled by control apparatus 18 in accordance with the parameters recited, inspiratory respiratory gas flow AF leaves humidification apparatus 38 with the setpoint moisture content. The inspiratory respiratory gas flow then enters second inspiration hose 36, which can be heated by conduit heating apparatus 37. The operation of conduit heating apparatus 37 is controllable by control apparatus 18 by means of a lead 88 in accordance with the temperature detected by proximal temperature sensor 48, in such a way that the proximal respiratory gas temperature detected by proximal temperature sensor 48 is above the dew point temperature of the humidified inspiratory respiratory gas flow AF.

The dew point temperature can be ascertained, based on the assumption that the respiratory gas exhibits the setpoint moisture content after passing through humidification apparatus 38, based on the proximal respiratory gas temperature detected by proximal temperature sensor 48 and further based on a dew point temperature curve stored in data memory 19. What is obtained from the dew point temperature curve is thus a setpoint temperature above which the temperature at proximal temperature sensor 48 should remain, preferably in fact should remain with a certain safety margin.

Storage of a dew point temperature curve in data memory 19 does not necessarily mean storage of a continuously differentiable curve. It is sufficient to store a dew point temperature curve that is approximated by a sufficient number of interpolation points, for example as a table. It is also possible to store, for the dew point temperature, an approximate mathematical function with which a dew point temperature can be ascertained based on a known absolute moisture content or on a known value pair of a relative moisture content and an associated temperature.

One problem in terms of properly supplying the patient with respiratory gas that is sufficiently humidified, but not over-humidified, is patient interface 31. Patient interface 31 is not heatable, as second inspiration hose 36 is, nor is it equipped with any sensors that might detect the ventilation state in patient interface 31. The reason for this is the usual nature of patient interface 31 as a disposable or single-use interface.

Specifically when ambient temperatures are very low, for instance in emergency situations, at the least the portion exposed to external environment U, between distal longitudinal end 31b of patient interface 31 and the entry of patient interface 31 into patient 12 at location 31c, is therefore greatly influenced by the ambient temperature. In this portion, patient interface 31 can be so severely cooled by the ambient temperature that an undesirable “rain out” occurs in patient interface 31. This undesired condensation of liquid out of inspiratory respiratory gas flow AF as a rule will occur most readily on the conduit wall of patient interface 31 which is cooled by ambient air. It is also not to be excluded, however, that respiratory gas flow AF in patient interface 31 cools to such an extent, under the influence of the ambient temperature, that fogging occurs in respiratory gas flow AF.

In order to avoid such undesired condensation of liquid in patient interface 31, control apparatus 18 is embodied to ascertain, on the basis of operating parameters of the ventilation apparatus which are transferred to it by the available sensors and in particular based on inspiratory respiratory gas flow AF, a resultant state, produced under the influence of the ambient temperature, of the ventilation situation in patient interface 31 downstream (in an inspiration direction) from coupling configuration 44a, and to determine on the basis of that ascertained resultant state whether condensation in patient interface 31 is to be expected.

For that purpose, either empirically ascertained data correlations or an analytically or numerically solvable equation system, constituting a model description of the thermal state of patient interface 31 during operation depending on operating data of ventilation apparatus 10 and its environment U, can be stored in data memory 19.

In a particularly simple and rapidly ascertainable calculation model, the resultant state of patient interface 31 is a resultant temperature of patient interface 31 which is ascertainable, for example, based on the proximal respiratory gas temperature at temperature sensor 48, in further consideration of the design configuration of patient interface 31 including the materials used therein, and in consideration of the ambient temperature measured by ambient temperature sensor 17. The inspiratory respiratory gas flow AF detected by proximal flow sensor 44, and if desired even an expiratory respiratory gas flow likewise detected by proximal flow sensor 44, can also be utilized in order to ascertain the resultant temperature.

Control apparatus 18, to which the dew point temperature of inspiratory respiratory gas flow AF is already available by way of the above-described control of ventilation apparatus 10, can thus assume that a risk of condensation in patient interface 31 is immediately imminent if a resultant temperature of patient interface 31 at a point located upstream in an inspiration direction from distal longitudinal end 31b (said temperature being ascertained based on an empirically ascertained data correlation and/or based on a thermodynamic model) falls below the dew point temperature. For the safety of the patient, it is preferably not the dew point temperature directly, but rather a threshold temperature that is a dew point temperature raised by a predetermined safety margin, that is utilized.

For the instance in which control apparatus 18 ascertains in this manner that condensation is definitely, or with a preponderant probability, or with more than a predetermined limit probability, occurring in patient interface 31, control apparatus 18 is embodied to decrease the operating power level delivered to evaporation apparatus 76. The amount by which the operating power level of evaporation apparatus 76 is decreased can be selected to be greater, the greater the condensation risk based on the ascertainment result, for example the greater the degree to which the resultant temperature of patient interface 31 quantitatively falls short of the threshold temperature or even the dew point temperature.

In addition, control apparatus 18 can also increase the operating power level of conduit heating arrangement 37 in order to increase the temperature of inspiratory respiratory gas flow AF on the way to proximal longitudinal end 30a of respiratory gas conduit arrangement 30. But because the present control apparatus 18 is intended to be able to prevent condensation of respiratory gas in patient interface 31 even for inspiration hoses that are not heatable, the decrease in the operating power level of evaporation apparatus 76 is implemented in all cases.

1. A ventilation apparatus for artificial ventilation of a patient, comprising:
a respiratory gas source arrangement that furnishes an inspiratory respiratory gas for artificial ventilation of the patient;
a flow modification apparatus that is embodied to generate and to quantitatively modify an inspiratory respiratory gas flow;
a humidification apparatus that is embodied to quantitatively increase the absolute moisture content of the inspiratory respiratory gas flow, the humidification apparatus comprising for that purpose a liquid reservoir and a modifiable-power-level evaporation apparatus;
a respiratory gas conduit arrangement having a proximal longitudinal end located closer to the patient during operation and having a distal longitudinal end located farther from the patient during operation, in order to convey the inspiratory respiratory gas flow from the humidification apparatus to the patient;
a proximal temperature sensor that is embodied to detect the temperature of the respiratory gas flow in the proximal longitudinal end region of the respiratory gas conduit arrangement;
a humidity sensor arrangement that is embodied to detect at least one humidity state value of the inspiratory respiratory gas flow which indirectly or directly represents an absolute moisture content of the inspiratory respiratory gas flow;
a flow sensor that is embodied to quantitatively detect the respiratory gas flow;
a control apparatus which is signal-transferringly connected to the proximal temperature sensor, to the humidity sensor arrangement, and to the flow sensor, and which is embodied to control the operating power level of the evaporation apparatus depending on a predefined setpoint moisture content of the respiratory gas and depending on signals of the proximal temperature sensor, of the humidity sensor arrangement, and of the flow sensor,
the humidity sensor arrangement being arranged upstream, in an inspiration direction of the respiratory gas flow, from the humidification apparatus and being embodied to detect the humidity state value upstream from the humidification apparatus; the ventilation apparatus comprising at least one ambient temperature sensor that is embodied to detect the temperature of the environment of the ventilation apparatus; the control apparatus being signal-transferringly connected to the at least one ambient temperature sensor and being embodied to control the operating power level of the evaporation apparatus, in addition to the dependences already recited, additionally depending on signals of the at least one ambient temperature sensor.

2. The ventilation apparatus according to claim 1, wherein it comprises an input apparatus for inputting a setpoint temperature of the respiratory gas flow in the proximal longitudinal end region of the respiratory gas conduit arrangement and/or for inputting a setpoint moisture content of the respiratory gas and/or for inputting a setpoint respiratory gas flow.

3. The ventilation apparatus according to claim 2, wherein it comprises an input apparatus for inputting a setpoint temperature of the respiratory gas flow in the proximal longitudinal end region of the respiratory gas conduit arrangement, and comprises a data memory which is queryable by the control apparatus and in which a correlation between temperature values of the respiratory gas flow in the proximal longitudinal end region of the respiratory gas conduit arrangement and associated setpoint moisture values of the respiratory gas is stored.

4. The ventilation apparatus according to claim 1, wherein at least one inspiratory conduit portion of the respiratory gas conduit arrangement comprises a conduit heating apparatus with which the inspiratory conduit portion is heatable.

5. The ventilation apparatus according to claim 1, wherein the humidity sensor arrangement is arranged downstream, in an inspiration direction of the respiratory gas flow, from the respiratory gas source arrangement.

6. The ventilation apparatus according to claim 1, wherein the control apparatus is signal-transferringly connected to the evaporation apparatus and/or to an energy supply thereof and/or to at least one energy sensor that is embodied to detect an energy delivered to the evaporation apparatus, in such a way that the energy and/or power level currently being delivered to the evaporation apparatus is ascertainable by the control apparatus from signals that are transferred via the signal-transferring connection.

7. The ventilation apparatus according to claim 1, wherein the respiratory gas conduit arrangement comprises at its proximal end a coupling configuration for coupling the respiratory gas conduit arrangement to a patient interface, for instance an endotracheal tube, a larynx mask, or a combination tube, and the like, which transfers respiratory gas between the respiratory gas conduit arrangement and the patient; the control apparatus being embodied to ascertain, depending on operating parameters of the ventilation apparatus, in particular of the respiratory gas flow, a resultant state of the ventilation situation downstream, in an inspiration direction, from the coupling configuration, and to modify the operating power level of the evaporation apparatus depending on the ascertained result.

8. The ventilation apparatus according to claim 7, wherein the resultant state of the ventilation situation encompasses a resultant temperature of the conduit wall of the patient interface and/or of the respiratory gas flow.

9. The ventilation apparatus according to claim 8, wherein it comprises a data memory which is queryable by the control apparatus and in which operating parameter values of the respiratory gas flow are associated with resultant temperature values of the conduit wall of the patient interface and/or of the respiratory gas flow.

10. The ventilation apparatus according to claim 7, wherein the control apparatus ascertains the resultant state depending on: the temperature, detected by the proximal temperature sensor, of the respiratory gas flow in the proximal longitudinal end region of the respiratory gas conduit arrangement; the magnitude of the respiratory gas flow detected by the flow sensor; and the predefined setpoint moisture content of the respiratory gas.

11. The ventilation apparatus according to claim 7, wherein the control apparatus ascertains the resultant state depending on the ambient temperature detected by the ambient temperature sensor.

12. The ventilation apparatus according to claim 7, wherein the control apparatus ascertains the resultant state for an ascertainment location located closer to the proximal end of the patient interface than to its distal end.

13. The ventilation apparatus according to claim 7, wherein the control apparatus ascertains the resultant state for a portion of the patient interface which is located between the coupling configuration and the entry point of the patient interface into the body of the patient.

14. The ventilation apparatus according to claim 7,
wherein at least one inspiratory conduit portion of the respiratory gas conduit arrangement comprises a conduit heating apparatus with which the inspiratory conduit portion is heatable, and
wherein the control apparatus is embodied to modify the temperature of the respiratory gas at the proximal temperature sensor, and for that purpose the operating power level of the conduit heating apparatus, depending on the ascertainment result.

15. The ventilation apparatus according to claim 4, wherein the control apparatus is embodied to control the operating power level of the conduit heating apparatus depending on a predefined setpoint temperature of the respiratory gas flow in the proximal longitudinal end region of the respiratory gas conduit arrangement.

16. The ventilation apparatus according to claim 12, wherein the control apparatus ascertains the resultant state for the proximal end of the patient interface.

17. The ventilation apparatus according to claim 14, wherein the control apparatus is embodied to control the operating power level of the conduit heating apparatus depending on a predefined setpoint temperature of the respiratory gas flow in the proximal longitudinal end region of the respiratory gas conduit arrangement.
